(19) United States
(12) Patent Application Publication
(10) Pub. No.: US 2025/0025497 A1
(43) Pub. Date: Jan. 23, 2025

(54) USE OF PHOSPHORUS-BASED MATERIAL IN PREPARATION OF MEDICAMENT FOR TREATING TUMORS

(71) Applicant: SHENZHEN INSTITUTES OF ADVANCED TECHNOLOGY, CHINESE ACADEMY OF SCIENCES, Shenzhen (CN)

(72) Inventors: Xuefeng YU, Yichang (CN); Wenhua ZHOU, Yichang (CN); Ting PAN, Yichang (CN); Haodong CUI, Yichang (CN)

(73) Assignee: SHENZHEN INSTITUTES OF ADVANCED TECHNOLOGY, CHINESE ACADEMY OF SCIENCES, Shenzhen (CN)

(21) Appl. No.: 18/910,172

(22) Filed: Oct. 9, 2024

Related U.S. Application Data
(63) Continuation of application No. 16/973,721, filed on Dec. 9, 2020.

(30) Foreign Application Priority Data
Jun. 13, 2018 (CN) 201810607846.X

(51) Int. Cl. A61K 33/42; A61P 35/00

(52) U.S. Cl. CPC A61K 33/42; A61P 35/00

(57) ABSTRACT

Use of phosphorus-based material in preparation of medicament for treating tumors. The phosphorus-based material is selected from a material which is convertible to produce phosphate ions in an acidic environment, and the phosphorus-based material can be converted by tumor cells phagocytosis to produce a large number of phosphate ions to change the intraceullar environment and extracellular environment, thereby inhibiting proliferation of tumor cells and inducing death of tumor cells. This process has no significant effect on normal cell activities. By applying the phosphorus-based material to the preparation of a medicament for treating tumor in a manner as mentioned above, the amplification and metastasis of tumor cells can be effectively inhibited, thereby the metastasis of the tumor cells and recurrence of the tumor can be prevented more effectively, improving the therapeutic effect of the tumor. The phosphorus-based material has little effect on normal cells and tissues in the course of treatment.

### CROSS-REFERENCE TO RELATED APPLICATIONS

The present application is a continuation of U.S. application Ser. No. 16/973,721, filed Dec. 9, 2020, which is a national phase entry under 35 USC § 371 of International Application PCT/CN2018/096460, filed Jul. 20, 2018, which claims the benefit of and priority to Chinese Patent Application No. 201810607846X, filed Jun. 13, 2018, the entire disclosures of which are incorporated herein by reference.

### TECHNICAL FIELD

The present invention relates to the technical field of medicament, and in particular, to use of phosphorus-based material in preparation of medicament for treating tumors.

### BACKGROUND

Intervention means like chemical medicament therapy, surgical therapy and radiation therapy combined with chemical medicament therapy are main manners of current clinical treatment of tumors and cancers. However, the chemotherapeutics molecules have a similar toxic effect on both cancer cells and normal cells, following severe toxic and side effects in the course of actual treatment, damaging human organs and hematopoietic function, some molecules may even cause alopecia, severe anemia, digestive dysfunction and impaired immune function etc. Meanwhile, the current treatment means and chemotherapeutics have not fundamentally and effectively solved the difficult problems in the treatment of cancers, such as recurrence, metastasis of cancers and severely impaired immune functions, which are still main causes of death.

Patent titiled “Selenium doped black phosphorus prodrug and preparation method thereof” (Application number CN107496444A), particularly discloses a prodrug in which selenium element is controllably released by using the selenium doped black phosphorus nanosheet as well as preparation method and use thereof; it constructs a two-dimensional black phosphorus loaded anti-tumor medicament, the surface of which is modified with polyoxyalkyleneamine, thereby realizing controllable release of selenium, and enhancing biocompatibility of the medicament. The black phosphorus nanosheet in this prodrug is used as a carrier of an anti-cancer medicament, its own anti-cancer effect has not been mentioned, and it has no targeting efficiency and specificity for a treatment of tumors.

Patent titled “Black phosphorus nanosheet and anti-tumor compound containing composite material and preparation method and use thereof” (Application number CN106267204A) specifically discloses a black phosphorus nanosheet and anti-tumor composite material with a primary amino group and/or a phenolic hydroxyl group as well as preparation method and use thereof, it constructs a two-dimensional black phosphorus loaded anti-tumor medicament, achieving a synergistic anti-cancer effect. Likewise, the black phosphorus nanosheet in this composite material is used as a carrier of an anti-cancer medicament, not the anti-cancer medicament, and the medicament has no targeting efficiency and specificity.

### SUMMARY

In order to improve the above-mentioned technical problems, the present invention aims at providing use of phosphorus-based material in preparation of medicament for treating tumors. The amplification and metastasis of tumor cells can be effectively inhibited, thereby the metastasis of the tumor cells and recurrence of the tumor can be prevented more effectively, improving the therapeutic effect of the tumor. In addition, the phosphorus-based material has little effect on normal cells and tissues in the course of treatment, being safe and reliable.

The technical solution adopted by the present invention is as follows: the preset invention provides use of phosphorus-based material in preparation of medicament for treating tumors, the phosphorus-based material is material which is convertible to produce phosphate ions in an acidic environment, and the phosphorus-based material can be converted by tumor cells phagocytosis to produce phosphate ions to change the intraceullar environment and extracellular environment, thereby inhibiting proliferation of the tumor cells and inducing death of the tumor cells.

Preferably, the phosphorus-based material is selected from elemental phosphorus and/or phosphorus-containing compound, which is convertible to produce phosphate ions in an acidic environment. The phosphorus-containing compound comprises phosphorous-containing oxide, phosphorous-containing halide and other phosphorus-containing compounds (such as phosphate, etc.) which is convertible to produce phosphate ions in an acidic environment. The phosphorus-based material specifically includes but is not limited to one or more of selected from elemental phosphorus such as black phosphorus, red phosphorus, white phosphorus, violet phosphorus etc; phosphorus-containing oxides such as phosphorus trioxide, phosphorus pentoxide, etc; phosphorus-containing halides such as phosphorus pentahalide, phosphorus trihalide, phosphorus tetrahalide, etc., and phosphate compounds which are based on orthophosphoric acid, metaphosphoric acid, phosphorous acid, pyrophosphoric acid, triphosphoric acid, hypophosphorous acid, hypophosphoric acid, and polyphosphoric acid, etc.

Preferably, the phosphorus-based material is phosphorus-based micro-nano material including nanometer-level and micrometer level material. Wherein, the nanometer-level material is beneficial for intravenous administration, reducing the cumulative organ toxicity; and the micrometer-level material is used for in situ administration, having a greater biological effect.

Preferably, the phosphorus-based material is subjected to surface modification for enhancing the targeting efficiency and/or stability. Wherein the surface modification for enhancing stability includes but is not limited to passive package modification based on liposome or polymer molecule, etc, ligand modification based on coordination bond, and the like; the surface modification for enhancing the targeting efficiency includes but is not limited to modification using compound like folic acid, etc, modification using peptide like cell penetrating peptides, etc, and modification of aptamer or antibody and the like targeting the cancer cells. By a targeting modification to surface of the phosphorus-based material, the amplification and metastasis of the cancer cells or tumor cells can be inhibited more effectively, thereby the recurrence of the cancer cells or the tumor cells can be prevented more effectively, further improving the therapeutic effect.

Preferably, the phosphorus-based material can be used as a single preparation; or the phosphorus-based material is used as an active ingredient, and a pharmaceutically acceptable adjuvant is added to jointly prepare a medicament for treating tumors; furthermore, other anti-tumor active ingredients can also be added, to achieve a synergistic effect. Specifically, a clinically acceptable dosage form can be made in accordance with the conventional processes, including tablets, capsules, pilulas, granules, sustained-release preparations, controlled-release preparations or injection preparations, etc, which are for clinical use. Wherein, the proportion of the phosphorus-based material in the combined medicament depends on the specific needs, the specific addition amount of the phosphorus-based material may be 0.01% to 99.99%, preferably 20% to 99.99%, further preferably 30% to 80%.

The above phosphorus-based material is used in the preparation of a medicament for treating tumors, the administration manner for the prepared medicament may be intravenous administration, or directly placed within the tumor and around the tumor. Specifically, the phosphorus-based material may be used in the preparation of a medicament for treating primary or secondary cancers and sarcomas or carcinosarcomas originated from brain, blood, mammary gland, pancreas, uterus, endometrium, uterine cervix, kidney, liver, gall bladder, head and neck, oral cavity, thyroid gland, skin, mucosa, gland, blood vessel, liver, lung, esophagus, ovary, prostate, osseous tissue, lymph node, urinary bladder, colon or rectum of human and animals. The tumor cells involved in the specific effects include but are not limited to human breast cancer cell MCF-7, human cervical cancer cell Hela, liver cancer cell HepG2, human non-small cell lung cancer cell A549, acute promyelocytic leukemia cell NB4, human brain glioma cell A172, human glioma cell LN-18, etc. The specific dose in the course of the treatment can be determined according to the type of the phosphorus-based material included in the medicament, the type of the targeted tumors and the administration manner, etc.

The beneficial technical effect of the present invention is: the present invention provides use of phosphorus-based material in the preparation of medicament for treating tumors, wherein the phosphorus-based material is a material which is convertible to produce phosphate ions in an acidic environment. The phosphorus-based material has a specific bioactivity, which is conversible to produce a large number of phosphate ions after being phagocytized by the tumor cells, changing the intraceullar environment and extracellular environment of the cells, and then inhibiting proliferation of the tumor cells and inducing death of the tumor cell. Specifically, due to an enhanced permeability and retention effect (EPR) of the tumor tissues and/or due to a targeting effect of its surface, etc., the phosphorus-based material is accumulated in the tumor tissue microenvironment, and/or after being ingested by the tumor cells via endocytosis, its conversion is accelerated due to the slightly acidic microenvironment inside and outside the tumor cells, and a large number of phosphate ions and other active products (i.e., unstable intermediate products, such as active radicals, reactive oxygen species, etc.) are instantaneously produced in the accelerated conversion process, named as “Ionic Bomb Effect”, further inducing a change in internal and external microenvironments of the tumor cells, and promoting a non-specific phosphorylation of proteins, thereby disrupting mitosis, inhibiting proliferation, and ultimately inducing death of the tumor cells. Meanwhile, for the normal cells, due to their slower division activity and slightly neutral intracellular and extracellular microenvironment, the conversion of the phosphorus-based material in the normal tissues and cells is slow, and the phosphate ions which are slowly released in this mild conversion process have a very high biocompatibility, resulting in a mere effect on the normal tissues and cells. In conclusion, the whole solution is simple and efficient, the phosphorus-based material has specificity and targeting efficiency in the course of treating cancers, this specific killing process against the tumor cells may be referred to as “Bioactive Phosphorus-based Therapy”, and abbreviated as “Bioactive Phosphorus-based Therapy (BPT)”.

### BRIEF DESCRIPTION OF THE DRAWINGS

In order to better illustrate the technical solutions in the embodiments of the preset invention, the accompanying drawings to be used in the description of embodiments will be briefly described below.

FIG. 1 shows a curve graph of inhibition on proliferation of breast cancer cell by the black phosphorus nanosheet in an embodiment of the present invention;

FIG. 2 shows a graph of breast cancer cell apoptosis induced by the black phosphorus nanosheet used in the embodiment of FIG. 1;

FIG. 3 shows a curve graph of inhibition on proliferation of cervical cancer cell by the black phosphorus nanosheet used in the embodiment of FIG. 1;

FIG. 4 shows a graph of cervical cancer cell apoptosis induced by the black phosphorus nanosheet used in the embodiment of FIG. 1;

FIG. 5 shows a curve graph of inhibition on the proliferation of non-small lung cancer cell by the black phosphorus nanosheet used in the embodiment of FIG. 1;

FIG. 6 shows a graph of non-small lung cancer cell apoptosisis induced by the black phosphorus nanosheet used in the embodiment of FIG. 1;

FIG. 7 shows a curve graph of inhibition on the proliferation of human normal cell by the black phosphorus nanosheet used in the embodiment of FIG. 1;

FIG. 8 shows a graph of human normal cell apoptosis induced by the black phosphorus nanosheet used in the embodiment of FIG. 1;

FIG. 9 shows a Raman scanning graph of gradual conversion of the black phosphorus nanosheet used in the embodiment of FIG. 1 phagocytized by human breast cancer cells in the cells.

### DETAILED DESCRIPTION

The preset invention will be further described as below with reference to specific examples. It should be understood that, these examples are only used to illustrate the invention rather than limit the scope of the invention. Furthermore, it should be understood that, after reading the content taught in the preset invention, a person skilled in the art can make various alterations or modifications to the invention, these equivalent forms likewise fall into the scope defined by the appended claims of the present application.

Firstly, a black phosphorus nanosheet having bioactivity was prepared by liquid phase stripping method. The specific steps comprise: in an air free environment, a certain amount of black phosphorus crystals were ground and dispersed in a solvent, then sealed, wherein a solvent could be various organic solvents, such as N-methyl pyrrolidone (NMP), dimethylformamide (DMF), dimethyl sulfoxide (DMSO), anhydrous ethanol, isopropanol, etc; then black phosphorus solution was stripped ultrasonically by means of probe ultrasound, water bath ultrasound or sequential action of the two, to prepare a black phosphorus sheet having bioactivity. The effect of the striping can be adjusted by changing an action mode of ultrasound and frequency of ultrasound, etc, the efficiency and yield of the stripping can also be improved in conjunction with other stripping techniques such as thermal separation technique, ion intercalation technique, etc.

The black phosphorus sheet was prepared according to the liquid phase stripping method as described above. Furthermore, the obtained black phosphorus sheet can also be subjected to a surface coordination or targeting modification, to enhance the stability and targeting efficiency of the black phosphorus sheet. The obtained two-dimensional black phosphorus (comprising the bared two-dimensional black phosphorus and modified two-dimensional black phosphorus) was dispersed in a suitable solvent, such as organic solvents of N-methyl pyrrolidone (NMP), dimethylformamide (DMF), dimethyl sulfoxide (DMSO), anhydrous ethanol, isopropanol, etc., to achieve a long-stem storage of the material.

Then the obtained black phosphorus nanosheet was applied on the tumor cells and the normal cells, to carry out a cell proliferation detection experiment and a cell apoptosis detection experiment. The specific steps are as follows:

### Experimental Method of Cell Proliferation Detection

Different types of human cancer cells (including cervical cancer cell, breast cancer cell and non-small lung cancer cell) and normal cells (choosing human bone marrow mesenchymal stem cell) were cultivated in advance. After the cells were counted, they were implanted in multi-well plates, culture medium was added, the cells were cultivated to grow. Specifically, the human cancer cell strains could be implanted into a 96-well plate at a density of 5000 cells/well, and four repeated wells were made for each group, the culture medium per well was 100 μL DMEM containing 10% FBS, the cells were placed in a 37° C. incubator, and cultivated under the condition of 5% CO2 and saturated humidity for 24 hours.

The black phosphorus sheet with proper size was chosen for washing, specifically the black phosphorus nanosheet with thickness of 2-10 nm and length and width of 20-300 nm was chosen to facilitate cell endocytosis. Then, it was diluted in accordance with a released dilution method, such that when the volume in the culture well was 100 μL, the concentration of the medicament was 0.125, 0.25, 0.5, 1, 2, 4, 8, and 16 μg/mL, respectively, the control group was a culture medium of medicament without addition of the black phosphorus sheet. The cells were placed in a 37° C. incubator, and cultivated under the condition of 5% CO2 and saturated humidity for 24 hours and 48 hours, then the original culture medium was discarded, and 100 μL of CCK8 working fluid was added and incubated for 1 hour, then the OD values were detected at A450 nm, the cell survival rate was calculated from the OD value of each well.

### (II) Experimental Method of Cell Apoptosis Detection

Different types of the human cancer cells (including cervical cancer cell, breast cancer cell and non-small lung cancer cell) and normal cells (choosing human mesenchymal stem cells) were cultivated in advance. After the cells were counted, they were implanted in multi-well plates, culture medium was added, the cells were cultivated to grow. Specifically, the human cancer cell strains were implanted into a 24-well plate at a density of 5×104 cells/well, and three repeated wells were made for each group, the culture medium per well was 1 mL DMEM containing 10% FBS, the cells were placed in a 37° C. incubator, and cultivated under the condition of 5% CO2 and saturated humidity for 24 hours.

The black phosphorus sheet with proper size was chosen for washing, specifically the black phosphorus nanosheet with thickness of 2-10 nm and length and width of 20-300 nm was chosen to facilitate cell endocytosis. Then, it was diluted in accordance with a released dilution method, such that when the volume in the culture well was 1 mL, the concentration of the medicament was 2, 4, and 8 μg/mL, respectively, and the control group was a culture medium of medicament without addition of the black phosphorus sheet. The cells were placed in a 37° C. incubator, and cultivated under the condition of 5% CO2 and saturated humidity for 48 hours, then the cells were trypsinized and collected, and centrifuged at 1000 g for 5 minutes, the supernatant was discarded, the cells were collected and gently resuspended in PBS, then counted. 50 to 100 thousand of the resuspended cells were taken, and centrifuged at 1000 g for 5 minutes, the supernatant was discarded, 195 μL of Annexin V-FITC binding buffer was added and the cells were gently resuspended, 5 μL of Annexin V-FITC and 10 μL of propidium iodide (PI) were added and gently mixed, stained under exclusion of light at room temperature for 15 minutes, and detected in a flow cytometer.

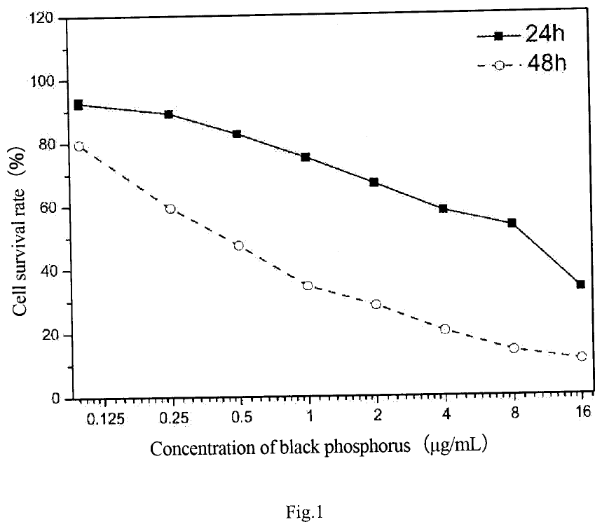

The black phosphorus nanosheets were subjected to an application study in vitro as described above, the obtained results are shown in FIGS. 1 to 8.

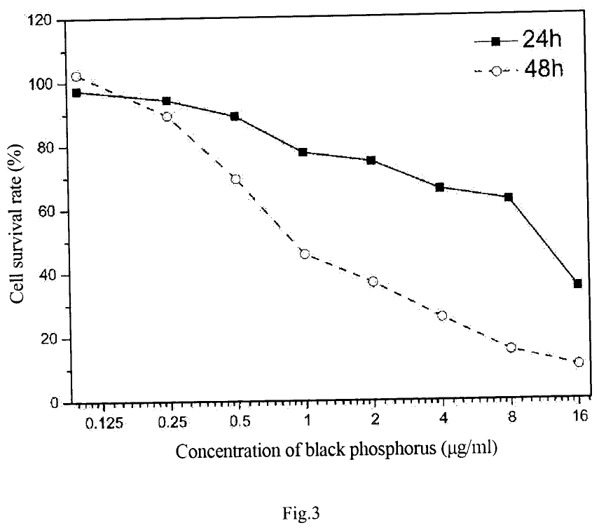

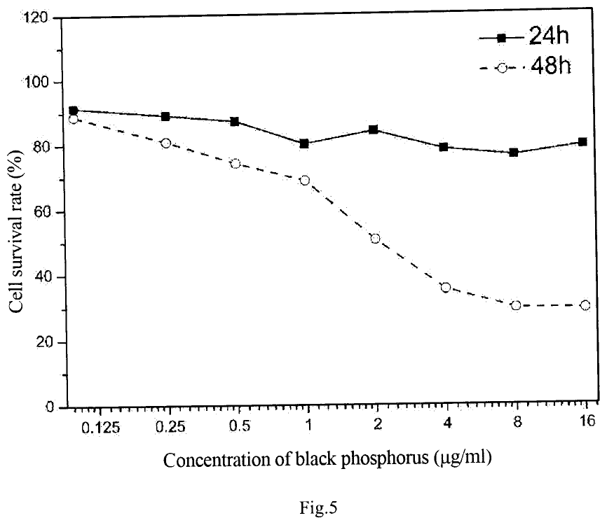

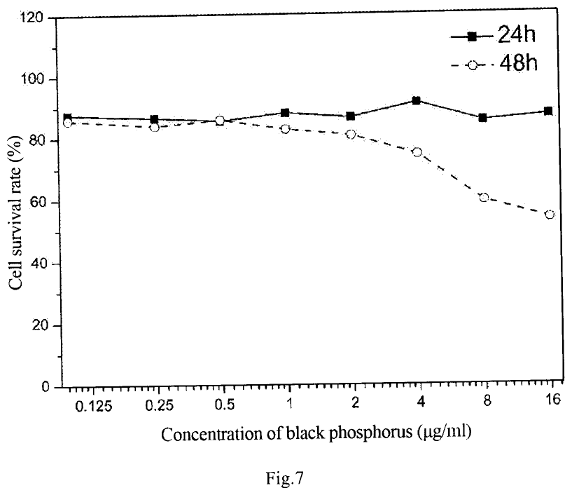

Please see FIGS. 1, 3, 5, and 7, FIG. 1 is the curve graph of inhibition on the proliferation of mammary gland cancer cell by the black phosphorus nanosheet in an embodiment of the preset invention, FIG. 3 is a curve graph of inhibition on proliferation of cervical cancer cell by the black phosphorus nanosheet, FIG. 5 is a curve graph of inhibition on the proliferation of non-small lung cancer cell by the black phosphorus nanosheet, FIG. 7 is a curve graph of inhibition on the proliferation of human normal cell by the black phosphorus nanosheet. In the above curve graphs of cell proliferation, the abscissa is the concentrations of the black phosphorus, and the ordinate is the survival rates of the cells.

As shown in FIGS. 1, 3, 5, and 7, in the cell proliferation detection experiment, the black phosphorus nanosheet was applied on three types of cancer cells: mammary gland cancer cell, cervical cancer cell and non-small lung cancer cell for 24 hours and 48 hours, then the black phosphorus nanosheet demonstrated concentration dependent cell proliferation effect to the three types of cancer cells, namely, the higher the concentration of the black phosphorus nanosheet was, the more evident inhibitory effect to the cancer cells was. After being cultivated for 48 hours, when the concentration of black phosphorus nanosheet was 0.5 μg/mL, the proliferation rate of the breast cancer cells was inhibited by about 50% (as shown in FIG. 1); when the concentration of the black phosphorus nanosheet was about 1 μg/mL, the inhibition rate on the proliferation of cervical cancer cells was up to 50% (as shown in FIG. 3); when the concentration of the black phosphorus nanosheet was about 2 μg/mL, the inhibition rate on the proliferation of non-small lung cancer cell was up to about 50% (as shown in FIG. 5). However, for the normal cells and the human mesenchymal stem cells, after being cultivated for 24 hours, the survival rates of the cells treated with the black phosphorus nanosheet at each concentration were all more than 90%. After being cultivated for 48 hours, the black phosphorus nanosheet at low concentration (less than 4 μg/mL) had no significant inhibition on the proliferation of the normal cells, and the survival rates of the cells ware more than 80% (as shown in FIG. 7). Thus, it can be seen that, the black phosphorus nanosheet could significantly inhibit the proliferation of cancer cells at lower dose; with the same dose, the inhibitory effect on the proliferation of normal cells by the black phosphorus nanosheet was far less than that on the cancer cells.

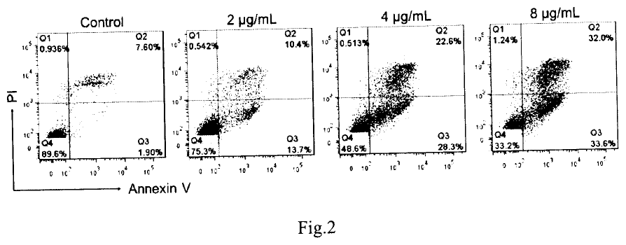

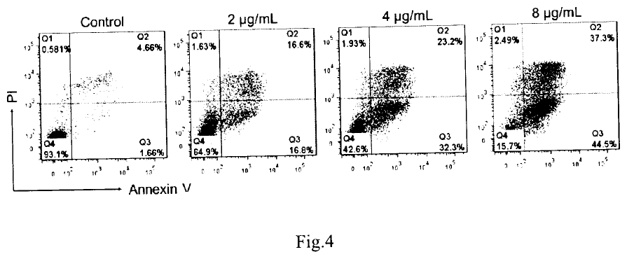

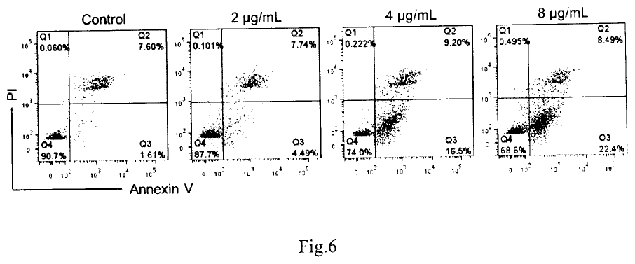

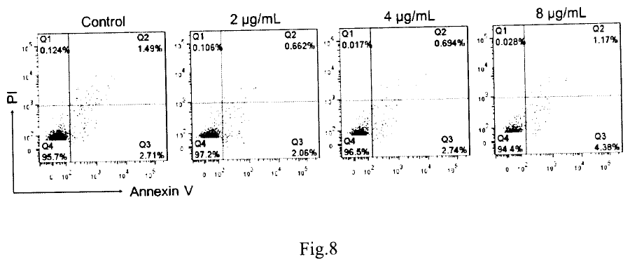

Please see FIGS. 2, 4, 6, and 8, FIG. 2 is a graph of breast cancer cell apoptosis induced by the black phosphorus nanosheet, FIG. 4 is a graph of cervical cancer cell apoptosis induced by the black phosphorus nanosheet, FIG. 6 is a graph of non-small lung cancer cell apoptosisis induced by the black phosphorus nanosheet, and FIG. 8 is a graph of human normal cell apoptosis induced by the black phosphorus nanosheet. In the above cell apoptosis graphs, the abscissa is fluorescence intensity of Annexin V; the ordinate is fluorescence intensity of PI; Q4 area is cells with normal activities; Q3 is early apoptotic cells; Q2 is late apoptotic cells; Q1 is necrotic cells, which can be ignored.

In the cell apoptosis detection experiment, when the concentration of the black phosphorus nanosheet was 2 μg/mL, the proportion of the normal cells (Q1) in the breast cancer cells was significantly reduced, the proportion of the apoptotic cell (Q2+Q3) was significantly increased, and with the increasing concentration of black phosphorus nonosheet, the proportion of the apoptotic cells was evidently increased; when the concentration of the black phosphorus nanosheet was 8 μg/mL, the proportion of the normal cell was decreased from 90% to 33% (as shown in FIG. 2). Likewise, when the concentration of the black phosphorus nanosheet was 2 μg/mL, the proportion of the normal cells (Q1) in the cervical cancer cell was decreased by 30%, the proportion of the apoptotic cell (Q2+Q3) was significantly increased; when the concentration of the black phosphorus nanosheet was 8 μg/mL, the proportion of the normal cells was decreased from from 93.1% to 15.7% (as shown in FIG. 4). For the non-small lung cancer cells, at a concentration of 8 μg/mL, the black phosphorus nanosheet could induce the proportion of the apoptotic cells to increase by about 21% (as show in FIG. 6). However, for the normal cells and and the human mesenchymal stem cells, after being treated with the black phosphorus nanosheet at each concentration for 48 hours, significant increase in the proportion of the apoptotic cells was not observed, the proportion of the normal cells in each group were all more than 94%. This proves that the black phosphorus nanosheet can effectively induce apoptosis of the cancer cells, meanwhile has no significant toxicity on the normal cells.

The above results prove that, the black phosphorus nanosheet can significantly and effectively inhibit the proliferation of the cancer cells, and induce their apoptosis, however, with the same dose, the killing effect against the normal cells was far less than that against the cancer cells. Such a selective killing effect against the tumor cells and the normal cancer cells by the black phosphorus nanosheet proves that the phosphorus-based material is very suitable to be used as the anti-cancer medicament in “Bioactive Phosphorus-based Therapy”, namely, the phosphorus-based material can be used in preparation of the medicament for treating tumors.

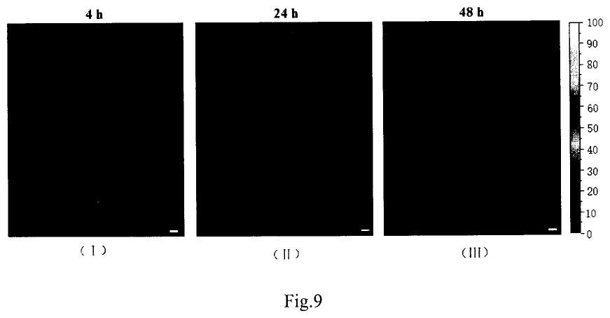

Besides, an observation experiment was also carried out for a conversion process of the black phosphorus nanosheet material in the cells, which is the above prepared black phosphorus nanosheet and applied to the human breast cancer cell (MCF-7), and then phagocytized by the cells. The results are shown in FIG. 9, FIG. 9 is a Raman scanning graph of gradual conversion of the black phosphorus nanosheet phagocytized by human breast cancer cells in the cells, and FIGS. (I), (II) and (III) represent the obtained Raman scanning graphs of the black phosphorus nano-material applied on MCF-7 cells for 4 hours, 24 hours and 48 hours, respectively, wherein, the fluorescence intensity detected was the signals of Raman characteristic peak A1g peak of the black phosphorus nanosheet. The signal of the peak, from small to large, indicates that the intensity of the A1g peak is from weak to strong, and the content of the black phosphorus nanosheet is from low to high. It can be known from FIG. 9 that when the black phosphorus nanosheet was applied to MCF-7 cell for 4 hours, a large number of signal peaks of the black phosphorus nanosheet could be observed in the cells, which were evidently reduced after 24 hours and could be rarely seen after 48 hours. This result proves that after being endocytised by cancer cells, the black phosphorus nanosheet can be rapidly converted in the cells.

It can be seen from the above that the black phosphorus nanosheet has bioactivity, which would be phagocytized by the tumor cells and converted to produce the phosphate ions in the tumor cells after contacting with the tumor cells. Due to the slightly acidic microenvironment inside and outside the tumor cells, a large number of phosphate ions and other active products (i.e., unstable intermediate products) are instantaneously produced in the accelerated conversion process, further inducing a change in internal and external microenvironments in the tumor cells, destroying the ionic equilibrium in the internal and external microenvironments of the tumor cells, and promoting a non-specific phosphorylation of proteins, thereby disrupting mitosis, inhibiting the proliferation, and ultimately inducing death of the tumor cells. According to the above action mechanism of the black phosphorus nanosheet against the tumor cells, other phosphorus-based materials, which is conversible to produce the phosphate ions in the acidic environment, including other elemental phosphorus and/or phosphorus-containing compounds, can also be used in preparation of the medicament for treating tumors, so as to act on the tumor cells through the above-mentioned similar action mechanism, and inhibit the proliferation of tumor cells and induce the death of tumor cells, thereby realizing the treatment of tumors.

Although the preset invention has been specifically shown and described with reference to the preferred embodiments, a skilled person in the art should understand that, various variations can be made to the preset invention in forms and details without departing from the spirit and scope of the preset invention as defined by the claims, they are all fall into the protection scope of the present invention.

1. A method of treating tumors consisting of administering a therapeutically effective amount of a medicament containing black phosphorus nanosheet.

2. The method of treating tumors according to claim 1, wherein the black phosphorus nanosheet is subjected to surface modification for enhancing targeting efficiency and/or stability.

3. The method of treating tumors according to claim 2, wherein the surface modification for enhancing stability is one or more selected from the group consisting of passive package modification based on liposomes or polymer molecules, ligand modification based on coordination bonding, and covalent modification.

4. The method of treating tumors according to claim 2, wherein the surface modification for enhancing the targeting efficiency is one or more selected from the group consisting of a compound modification, a peptide modification, and a modification of an aptamer or antibody targeting the cancer cells.

5. The method of treating tumors according to claim 1, wherein the addition amount of the black phosphorus nanosheet used in the medicament containing black phosphorus nanosheet is 1% to 99.99%, preferably 30% to 80%.

6. The method of treating tumors according to claim 1, wherein the tumor is selected from breast cancer, cervical cancer, lung cancer, liver cancer, and brain glioma.

7. The method of treating tumors according to claim 1, wherein the black phosphorus nanosheet is used as a single active ingredient.

8. The method of treating tumors according to claim 1, wherein the black phosphorus nanosheet is used as a single preparation.

9. The method of treating tumors according to claim 1, wherein the concentration of the black phosphorus nanosheet is 2-8 μg/mL.

10. The method of treating tumors according to claim 1, wherein the medicament containing black phosphorus nanosheet further comprises a pharmaceutically acceptable adjuvant.

11. A method of treating tumors comprising administering a therapeutically effective amount of a medicament containing black phosphorus nanosheet, wherein due to an enhanced permeability and retention effect of tumor tissues and/or a targeting effect of the black phosphorus nanosheet, the black phosphorus nanosheet is ingested by tumor cells via endocytosis, accumulated in a tumor tissue microenvironment, and converted to produce phosphate ions due to the slightly acidic microenvironment inside and outside the tumor cells, which induces a change in internal and external microenvironments of the tumor cells, and promotes a non-specific phosphorylation of proteins, thereby disrupting mitosis, inhibiting proliferation, and inducing death of the tumor cells.

12. The method of treating tumors according to claim 11, wherein the black phosphorus nanosheet is subjected to surface modification for enhancing targeting efficiency and/or stability.

13. The method of treating tumors according to claim 11, wherein the surface modification for enhancing stability is one or more selected from the group consisting of passive package modification based on liposomes or polymer molecules, ligand modification based on coordination bonding, and covalent modification.

14. The method of treating tumors according to claim 11, wherein the surface modification for enhancing the targeting efficiency is one or more selected from the group consisting of a compound modification, a peptide modification, and a modification of an aptamer or antibody targeting the cancer cells.

15. The method of treating tumors according to claim 11, wherein the addition amount of the black phosphorus nanosheet used in the medicament containing black phosphorus nanosheet is 1% to 99.99%, preferably 30% to 80%.

16. The method of treating tumors according to claim 11, wherein the tumor is selected from breast cancer, cervical cancer, lung cancer, liver cancer, and brain glioma.

17. The method of treating tumors according to claim 11, wherein the black phosphorus nanosheet is used as a single active ingredient.

18. The method of treating tumors according to claim 11, wherein the black phosphorus nanosheet is used as a single preparation.

19. The method of treating tumors according to claim 11, wherein the concentration of the black phosphorus nanosheet is 2-8 μg/mL.

20. The method of treating tumors according to claim 11, wherein the medicament containing black phosphorus nanosheet further comprises a pharmaceutically acceptable adjuvant.
